(19) United States
(12) Patent Application Publication
(10) Pub. No.: US 2024/0197748 A1
(43) Pub. Date: Jun. 20, 2024

(54) COMBINATION TREATMENT FOR MELANOMA

(71) Applicant: AstraZeneca AB, Södertälje (SE)

(72) Inventors: Simon Andrew SMITH, Cambridge (GB); Emma Jane DEAN, Cambridge (GB); Jeeyun LEE, Seoul (KR)

(21) Appl. No.: 18/552,300

(22) PCT Filed: Mar. 25, 2022
(86) PCT No.: PCT/EP2022/057895

§ 371 Date: Sep. 25, 2023

Related U.S. Application Data
(60) Provisional application No. 63/166,295, filed on Mar. 26, 2021.
(60) Provisional application No. 63/208,728, filed on Jun. 9, 2021.

(51) Int. Cl. A61K 31/5377; A61K 39/395; A61P 35/04

(52) U.S. Cl. CPC A61K 31/5377; A61K 39/3955; A61P 35/04

(57) ABSTRACT

Combination Treatments for Melanoma This specification discloses the use of an ATR inhibitor in combination with an immune checkpoint inhibitor for the treatment of melanoma in a patient who has previously received immunotherapy.

### FIELD

The present specification relates to a combination of an ATR inhibitor such as 4-{4-[(3R)-3-methylmorpholin-4-yl]-6-[1-((R)-S-methylsulfonimidoyl)cyclopropyl]pyrimidin-2-yl}-1H-pyrrolo[2,3-b]pyridine (AZD6738, ceralasertib, Compound (1) below or a pharmaceutically acceptable salt thereof) and an immune checkpoint inhibitor such as durvalumab for use in the treatment of melanoma, where the combination is administered to a patient who has previously received immunotherapy. The specification also relates to methods of treatment of melanoma involving the administration of an ATR inhibitor in combination with an immune checkpoint inhibitor to a patient who has previously received immunotherapy, the use of an ATR inhibitor in combination with an immune checkpoint inhibitor for the manufacture of a medicament for the treatment of melanoma in patients who have previously received immunotherapy.

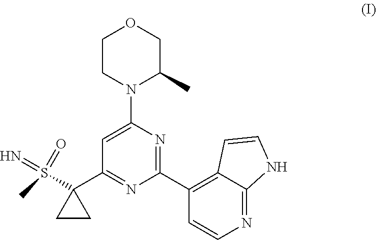 [SMILES: C[C@@H]1COCCN1c1cc(C2([S@](C)(=N)=O)CC2)nc(-c2ccnc3[nH]ccc23)n1]

### BACKGROUND

ATR is a serine/threonine protein kinase and member of the phosphatidylinositol 3-kinase related kinase (PIKK) family. During normal DNA replication, ATR is recruited at stalled replication forks, which can progress to double strand breaks if left unrepaired. ATR is also recruited to single strand DNA coated with Replication Protein A (RPA) following single strand DNA damage or the resection of double strand breaks. Recruitment and activation of ATR leads to cell cycle arrest in the S-phase while the DNA is repaired and the stalled replication fork resolved, or nuclear fragmentation and entry into programmed cell death (apoptosis).

As a result, ATR inhibitors are expected to cause growth inhibition in tumour cells dependent upon ATR for DNA repair e.g. ATM-deficient tumours. In addition to such monotherapy activity, ATR inhibitors are also predicted to potentiate the activity of cytotoxic DNA damaging agents and radiotherapy (through inhibition of ATR-dependent DNA repair processes) when used in combination.

Example ATR inhibitors include AZD6738, a potent inhibitor of ATR with good selectivity against other PIKK family members first disclosed in WO2011/154737. This compound is being developed as an oral anti-tumour agent in patients with disease that is dependent upon ATR function for DNA repair, for example tumours that are deficient of the serine/threonine-specific protein kinase, ATM. AZD6738 is being investigated in clinical trials against various forms of cancer.

Melanoma develops from the pigment-producing cells known as melanocytes in the skin and is the most dangerous form of skin cancer. In 2015, there were 3.1 million people with active melanoma, which resulted in 59,800 deaths (Vos et al., Lancet 388, 1545-1602); while in 2020 the American Cancer Society estimates about 100,000 new cases will be diagnosed locally, with around 7,000 deaths.

The current standard of care for melanoma is based on first-line immunotherapy, for example using immune checkpoint inhibitors such as nivolumab or pembrolizumab. Patients with actionable mutations, such as those with BRAF mutations may receive targeted agents. Patients may receive several different lines of immunotherapy but once it is no longer effective, standard chemotherapy like doublet carboplatin and paclitaxel or single agent paclitaxel may be used to continue treatment. However, response to chemotherapy is often poor with about 20% responding to doublet chemotherapy and only around 5% of patients responding to taxanes. There is therefore a pressing need for additional approaches that can be used to treat resistant melanoma cancers which are no longer amenable to immunotherapy.

### SUMMARY

Surprisingly and unexpectedly, through the analysis of phase II clinical trial data, it has been found that ATR inhibitors such as AZD6738 in combination with immune checkpoint inhibitor such as durvalumab are effective in melanoma patients who have previously received treatment with immunotherapy, with a high proportion of such individuals responding to subsequent ATR inhibition and immunotherapy. In particular, the combination provides improved progression free survival, overall survival and improved duration of response. Responses were observed across all the melanoma subtypes enrolled, even in acral and mucosal melanoma which are known to be harder to treat. Furthermore, the combination was found to be effective in patients with primary resistance (as well as secondary resistance) which is usually harder to treat. Melanoma patients who have received prior immunotherapy have therefore been identified as a target population for treatment with a combination of ATR inhibitors such as AZD6738 and immune checkpoint inhibitors such as the PD-L1 inhibitor durvalumab.

Without wishing to be bound by theory, it is believed that ATR inhibitors reset the immune profile of the melanoma, thereby sensitising the melanoma to further treatment with immunotherapy.

It is an object of the present specification to provide a combination of an ATR inhibitor such as AZD6738 and an immune checkpoint inhibitor such as durvalumab for the treatment of melanoma in patients who have previously received immunotherapy.

In an aspect of the present specification there is provided a combination comprising an ATR inhibitor and an immune checkpoint inhibitor for use in the treatment of melanoma in a patient who has previously received immunotherapy.

In a further aspect of the present specification there is provided a method of treating melanoma in a human or animal patient, who has previously received immunotherapy, in need of such treatment, comprising administering to said patient an effective amount of an ATR inhibitor in combination with an effective amount of an immune checkpoint inhibitor.

In a further aspect of the present specification there is provided the use of an ATR inhibitor, in combination with an immune checkpoint inhibitor, in the manufacture of a medicament for the treatment of melanoma, where the patient has previously received immunotherapy.

In a further aspect of the present specification there is provided a combination treatment comprising the administration of an effective amount of an ATR inhibitor, optionally together with a pharmaceutically acceptable diluent or carrier, in combination with an effective amount of an immune checkpoint inhibitor, optionally together with a pharmaceutically acceptable diluent or carrier to a human or animal patient in need of such therapeutic treatment that has received immunotherapy, for use in the treatment of melanoma.

In a further aspect of the present specification there is provided a kit comprising a pharmaceutical composition comprising an ATR inhibitor and at least one pharmaceutically acceptable excipient, and a pharmaceutical composition comprising an immune checkpoint inhibitor, and instructions for the use of the pharmaceutical compositions in the treatment of melanoma, where the pharmaceutical compositions are administered to a patient who has previously received immunotherapy.

### FIGURES

FIG. 1: Bar chart showing melanoma patient responses to combined AZD6738 and durvalumab treatment.

FIG. 2: Swimmer plot showing duration of AZD6738 and durvalumab treatment for melanoma patients along with patient response. The “x” on the bars corresponds to the date of first response with duration of response being calculated from x. Patient P30 had progressive disease (PD) and no bar is shown since the patient progressed after 10 days and therefore the start date of administration and the last date of administration were the same.

### DETAILED DESCRIPTION

The invention detailed in this specification should not be interpreted as being limited to any of the recited embodiments or examples. Other embodiments will be readily apparent to a reader skilled in the art.

“A” or “an” mean “at least one”. In any embodiment where “a” or “an” are used to denote a given element, “a” or “an” may mean one. In any embodiment where “a” or “an” are used to denote a given element, “a” or “an” may mean 1, 2, 3, 4, 5, 6, 7, 8, 9 or 10.

When an embodiment includes “a” or “an” element X, subsequent referrals to “the” element X do not imply only one of the element is present. Instead the above interpretation of “a” or “an” continues to apply so that “the” also means “at least one”. In other words, embodiments comprising “an element X, where the element X is . . . ” should be construed as “at least one element X, where the at least one element X is . . . ”.

“Comprising” means that a given material or element may contain other materials or elements. In any embodiment where “comprising” is mentioned the given material or element may be formed of at least 10% w/w, at least 20% w/w, at least 30% w/w, or at least 40% w/w of the material or element (or combination of materials or elements). In any embodiment where “comprising” is mentioned, “comprising” may also mean “consisting of” (or “consists of”) or “consisting essentially of” (or “consists essentially of”) a given material or element.

“Consisting of” or “consists of” means that a given material or element is formed entirely of the material or element (or combination of materials or elements). In any embodiment where “consisting of” or “consists of” is mentioned the given material or element may be formed of 100% w/w of the material or element.

“Consisting essentially of” or “consists essentially of” means that a given material or element consists almost entirely of that material or element (or combination of materials or elements). In any embodiment where “consisting essentially of” or “consists essentially of” is mentioned the given material or element may be formed of at least 50% w/w, at least 60% w/w, at least 70% w/w, at least 80% w/w, at least 90% w/w, at least 95% w/w or at least 99% w/w of the material or element.

In any embodiment where “is” or “may be” is used to define a material or element, “is” or “may be” may mean the material or element “consists of” or “consists essentially of” the material or element.

When it is mentioned that “in some embodiments . . . ” a certain element may be present, the element may be present in a suitable embodiment in any part of the specification, not just a suitable embodiment in the same section or textual region of the specification.

When a feature is “selected from” a particular list, the feature may be selected from a list consisting of the specified alternatives (i.e. a list of those alternatives and no others).

Claims are embodiments.

### Therapeutic Use

In one embodiment there is provided a combination of an ATR inhibitor and an immune checkpoint inhibitor for use in the treatment of melanoma, where the combination is administered to a patient who has previously received immunotherapy.

In one embodiment there is provided a method of treatment of melanoma in a human or animal patient in need of such treatment, comprising administering to said patient an effective amount of an ATR inhibitor in combination with an effective amount of an immune checkpoint inhibitor wherein the patient has previously received immunotherapy.

In one embodiment there is provided the use of an ATR inhibitor in combination with an immune checkpoint inhibitor, in the manufacture of a medicament for the treatment of melanoma, where the medicament is administered to a patient who has previously received immunotherapy.

Where the term “combination” is used it is to be understood that this refers to simultaneous, separate or sequential administration.

In some embodiments, a combination may be simultaneously, separately and/or sequentially administered.

In some embodiments, a combination may be simultaneously administered.

In some embodiments, a combination may be separately administered.

In some embodiments, a combination may be sequentially administered.

Where the administration of a combination is sequential or separate, the delay in administering the second component should not be such as to lose the beneficial effect of the combination.

In one embodiment, the ATR inhibitor may be administered before the immune checkpoint inhibitor. For example, the ATR inhibitor may be administered 2 hours, 4 hours, 6 hours, 8 hours, 10 hours, 12 hours, 16 hours, 24 hours, or 48 hours before the immune checkpoint inhibitor.

In one embodiment, the ATR inhibitor may be administered after the immune checkpoint inhibitor. For example, the ATR inhibitor may be administered 2 hours, 4 hours, 6 hours, 8 hours, 10 hours, 12 hours, 16 hours, 24 hours, or 48 hours after the immune checkpoint inhibitor.

### Immunotherapy

Where a patient has “previously received immunotherapy”, this includes patients who have been successfully or unsuccessfully treated with immunotherapy, such that their cancer responded or did not respond to treatment respectively. Patients who have previously received immunotherapy may have halted previous treatment due to treatment failure, where the cancer growth or health impact of the disease is not, or is no longer, positively managed by the immunotherapy. Where such a treatment has failed, the cancer may be described as resistant to immunotherapy. Primary resistance occurs when some inherent characteristic of the cancer prevents the immunotherapy from working whereas acquired resistance, also known as secondary resistance, occurs when the cancer becomes resistant during immunotherapy treatment. Some patients may receive immunotherapy as an adjuvant therapy. Patients who relapse on adjuvant immunotherapy may also be considered to have primary resistance to immunotherapy.

In some embodiments, the patient's cancer may be resistant to immunotherapy. In some embodiments the patient's cancer may be resistant to PD-1 inhibitor, PD-L1 inhibitor or CTLA-4 inhibitor immunotherapy. In some embodiments the patient's cancer may be resistant to PD-1 inhibitor immunotherapy. In some embodiments the patient's cancer may be resistant to PD-L1 inhibitor immunotherapy. In some embodiments the patient's cancer may be resistant to CTLA-4 inhibitor immunotherapy.

In some embodiments, the patient has primary resistance to immunotherapy. In some embodiments the patient has primary resistance to PD-1 inhibitor, PD-L1 inhibitor or CTLA-4 inhibitor immunotherapy. In some embodiments the patient has primary resistance to PD-1 inhibitor immunotherapy. In some embodiments the patient has primary resistance to PD-L1 inhibitor immunotherapy. In some embodiments the patient has primary resistance to CTLA-4 inhibitor immunotherapy. In one embodiment, primary resistance is defined according to the Society for Immunotherapy of Cancer (SITC) recommendations as having >6 weeks of immunotherapy drug exposure and a best response of progressive disease or stable disease for less than 6 months before progressing.

In some embodiments, the patient has acquired (or secondary) resistance to immunotherapy. In some embodiments the patient has acquired resistance to PD-1 inhibitor, PD-L1 inhibitor or CTLA-4 inhibitor immunotherapy. In some embodiments the patient has acquired resistance to PD-1 inhibitor immunotherapy. In some embodiments the patient has acquired resistance to PD-L1 inhibitor immunotherapy. In some embodiments the patient has acquired resistance to CTLA-4 inhibitor immunotherapy. In one embodiment, acquired resistance is defined according to SITC recommendations as having 6 weeks of immunotherapy drug exposure and a best response of complete response, partial response or stable disease for more than 6 months before progressing.

“Immunotherapy” is the use of a patient's own immune system to treat disease, for example cancer. It includes stimulating the natural defences of a patient's immune system so it is better at finding and attacking harmful species in the body (for example cancer cells), as well as administering drugs that act like immune system components to restore or improve how the immune system works to defend the body (for example to find and attack cancer cells).

In some embodiments, the patient has received at least one form of immunotherapy for a minimum of 6 weeks prior to treatment with the combinations described herein. In some embodiments immunotherapy may comprise treatment with an immune checkpoint inhibitor, chimeric antigen receptor T-cell therapy, treatment with a cytokine, treatment with an immunomodulator, treatment with a cancer vaccine, treatment with a monoclonal antibody and/or treatment with an oncolytic virus.

“Checkpoint inhibitors” include any substance which blocks immune checkpoints: key regulators of the immune system that when stimulated can dampen the immune response to an immunologic stimulus. Some cancers can protect themselves from attack by stimulating immune checkpoint targets, so checkpoint therapy is used that can block inhibitory checkpoints, restoring immune system function.

Example checkpoint inhibitors include PD-1 inhibitors (for example pembrolizumab [Keytruda®], nivolumab [Opdivo®], cemiplimab [Libtayo®], spartalizumab [PDR001], camrelizumab [SHR1210], sintilimab [1B1308], tislelizumab [BGB-A317], toripalimab [JS 001], AMP-224 or AMP-514), PD-L1 inhibitors (for example atezolizumab [Tecentriq®], avelumab [Bavencio®], durvalumab [MED14736, Imfinzi®], KN035, CK-301, AUNP12, CA-170 or BMS-986189) and CTLA-4 inhibitors (for example ipilimumab [Yervoy®] or tremelimumab).

In some embodiments immunotherapy may comprise treatment with an immune checkpoint inhibitor.

In some embodiments immunotherapy may comprise treatment with an immune checkpoint inhibitor selected from a PD-1 inhibitor, a PD-L1 inhibitor and a CTLA-4 inhibitor.

In some embodiments immunotherapy may comprise treatment with an immune checkpoint inhibitor which is a PD-1 inhibitor.

In some embodiments immunotherapy may comprise treatment with an immune checkpoint inhibitor which is a PD-L1 inhibitor.

In some embodiments immunotherapy may comprise treatment with an immune checkpoint inhibitor which is a CTLA-4 inhibitor.

In some embodiments immunotherapy may comprise treatment with an immune checkpoint inhibitor selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, BMS-986189, ipilimumab and tremelimumab.

In some embodiments immunotherapy may comprise treatment with an immune checkpoint inhibitor selected from the group consisting of pembrolizumab, nivolumab, cemiplimab, atezolizumab, avelumab, durvalumab and ipilimumab.

In some embodiments immunotherapy may comprise treatment with an immune checkpoint inhibitor selected from pembrolizumab and nivolumab.

In some embodiments immunotherapy may comprise chimeric antigen receptor T-cell therapy.

“Chimeric antigen receptor (CAR) T-cell therapy” takes some T-cells from a patient's blood, mixes them with a special virus that makes the T-cells learn how to attach to tumour cells, and then gives the cells back to the patient so they can find, attach to, and kill cancer.

In some embodiments immunotherapy may comprise treatment with a cytokine.

“Cytokines” are small proteins that carry messages between cells and stimulate immune cells to attack cancer.

In some embodiments immunotherapy may comprise treatment with an immunomodulator.

“Immunomodulators” are drugs that generally boost parts of the immune system to treat certain types of cancer.

In some embodiments immunotherapy may comprise treatment with a cancer vaccine.

“Cancer vaccines” are substances put into the body to start an immune response against cancer. They can be used prophylactically or to increase a body's immune response, allowing more effective treatment.

In some embodiments immunotherapy may comprise treatment with a monoclonal antibody.

“Monoclonal antibodies” (mAbs or MoAbs) are man-made versions of immune system proteins. Monoclonal antibodies can be designed to attack a very specific part of a cancer cell.

In some embodiments immunotherapy may comprise treatment with an oncolytic virus.

“Oncolytic virus” treatment uses viruses that have been modified in a lab to infect and kill certain tumour cells.

In some embodiments immunotherapy may comprise treatment with one immunotherapy agent.

In some embodiments immunotherapy may comprise treatment with more than one immunotherapy agent, for example, a PD-L1 or PD-1 antibody in combination with a CTLA-4 antibody, such as nivolumab in combination with ipilimumab.

### ATR Inhibitors

An “ATR inhibitor” is any compound which attenuates the activity of the ATR enzyme in-vitro or in-vivo. ATR inhibitors may be selective or unselective, small molecules or biomolecules.

Example ATR inhibitors include AZD6738, M6620 (berzosertib), BAY-1895344, EPT-46464, VE-821 and VX-970.

In some embodiments an ATR inhibitor may be selected from the group consisting of AZD6738, M6620, BAY-1895344, EPT-46464, VE-821 and VX-970.

In some embodiments an ATR inhibitor may be AZD6738.

In some embodiments AZD6738 may be Compound (I) or a pharmaceutically acceptable salt thereof.

The term “pharmaceutically acceptable” is used to specify that an object (for example a salt, dosage form or excipient) is suitable for use in patients and/or has clinical or commercial precedence.

An example list of pharmaceutically acceptable salts can be found in the “Handbook of Pharmaceutical Salts: Properties, Selection and Use”, P. H. Stahl and C. G. Wermuth, editors, Weinheim/Zurich: Wiley-VCH/VFiCA, 2002 or subsequent editions.

In some embodiments AZD6738 may be Compound (I) in a salt-free form (for example in a neutral or zwitterionic form, or for example in a free base or free acid form).

In some embodiments AZD6738 may be a pharmaceutically acceptable salt of Compound (I).

A suitable pharmaceutically acceptable salt of Compound (I) is, for example, an acid-addition salt. An acid addition salt of Compound (I) may be formed by bringing the compound into contact with a suitable inorganic or organic acid under conditions known to the skilled person.

An acid addition salt may for example be formed using an inorganic acid selected from hydrochloric acid, hydrobromic acid, sulphuric acid and phosphoric acid. An acid addition salt may also be formed using an organic acid selected from citric acid, fumaric acid, maleic acid and methane sulfonic acid.

A further suitable pharmaceutically acceptable salt of Compound (I) is, for example, a salt formed within the human or animal body after administration of Compound (I) to said human or animal body.

In some embodiments an ATR inhibitor may be AZD6738 which is administered according to a two weeks on/two weeks off (14 days on/14 days off) schedule within a 28-day cycle.

In some embodiments an ATR inhibitor may be AZD6738 which is administered for 14 consecutive days within a 28-day cycle.

In some embodiments an ATR inhibitor may be AZD6738 which is administered on days 1 to 14 of a 28-day cycle.

In some embodiments an ATR inhibitor may be AZD6738 which is administered on days 15 to 28 of a 28-day cycle.

In some embodiments an ATR inhibitor may be AZD6738 which is administered according to a one week on/three weeks off (7 days on/21 days off) schedule within a 28-day cycle.

In some embodiments an ATR inhibitor may be AZD6738 which is administered for 7 consecutive days within a 28-day cycle.

In some embodiments an ATR inhibitor may be AZD6738 which is administered on days 1 to 7 of a 28-day cycle.

A “28-day cycle” is a single treatment period which may be continuously repeated for a given patient, or may be repeated with a treatment gap (of for example 1, 2, 3, 4, 5, 6, 7, 8, 9, 10, 11, 12, 13, 14, 30 or 60 days) between discrete cycles.

In some embodiments an ATR inhibitor may be AZD6738 which is administered in a total daily dose between 30 mg and 500 mg.

In some embodiments an ATR inhibitor may be AZD6738 which is administered in a total daily dose of 40 mg, 60 mg, 80 mg, 160 mg, 240 mg, 320 mg or 480 mg.

In some embodiments an ATR inhibitor may be AZD6738 which is administered in a total daily dose of 480 mg.

In some embodiments an ATR inhibitor may be AZD6738 which is administered twice daily in 240 mg doses (i.e. in a 480 mg total daily dose, administered in two separate tranches each consisting of 50% of the total daily dose, also referred to as 240 mg BID).

In some embodiments an ATR inhibitor may be AZD6738 which is administered in a total daily dose of 320 mg.

In some embodiments an ATR inhibitor may be AZD6738 which is administered twice daily in 160 mg doses (i.e. in a 160 mg total daily dose, administered in two separate tranches each consisting of 50% of the total daily dose, also referred to as 160 mg BID).

In some embodiments an ATR inhibitor may be AZD6738 which is administered twice daily on a two weeks on/two weeks off schedule within a 28-day cycle.

In some embodiments an ATR inhibitor may be AZD6738 which is administered twice daily in 240 mg doses on a two weeks on/two weeks off schedule within a 28-day cycle.

In some embodiments an ATR inhibitor may be AZD6738 which is administered twice daily in 240 mg doses for 14 consecutive days within a 28 day cycle.

In some embodiments an ATR inhibitor may be AZD6738 which is administered twice daily in 240 mg doses on days 1 to 14 of a 28-day cycle.

In some embodiments an ATR inhibitor may be AZD6738 which is administered twice daily in 240 mg doses on days 15 to 28 of a 28-day cycle.

In some embodiments an ATR inhibitor may be AZD6738 which is administered twice daily in 160 mg doses on a two weeks on/two weeks off schedule within a 28-day cycle.

In some embodiments an ATR inhibitor may be AZD6738 which is administered twice daily in 160 mg doses for 14 consecutive days within a 28 day cycle.

In some embodiments an ATR inhibitor may be AZD6738 which is administered twice daily in 160 mg doses on days 1 to 14 of a 28-day cycle.

In some embodiments an ATR inhibitor may be AZD6738 which is administered twice daily in 160 mg doses on days 15 to 28 of a 28-day cycle.

In some embodiments an ATR inhibitor may be AZD6738 which is administered twice daily on a one week on/three weeks off (7 days on/21 days off) schedule within a 28-day cycle.

In some embodiments an ATR inhibitor may be AZD6738 which is administered twice daily in 240 mg doses on a one week on/three weeks off schedule within a 28-day cycle.

In some embodiments an ATR inhibitor may be AZD6738 which is administered twice daily in 240 mg doses for 7 consecutive days within a 28-day cycle.

In some embodiments an ATR inhibitor may be AZD6738 which is administered as a 240 mg dose twice daily on days 1 to 7 of a 28-day cycle.

In some embodiments an ATR inhibitor may be AZD6738 which is administered twice daily in 160 mg doses on a one week on/three weeks off schedule within a 28-day cycle.

In some embodiments an ATR inhibitor may be AZD6738 which is administered twice daily in 160 mg doses for 7 consecutive days within a 28-day cycle.

In some embodiments an ATR inhibitor may be AZD6738 which is administered as a 160 mg dose twice daily on days 1 to 7 of a 28-day cycle.

### Immune Checkpoint Inhibitors

A “checkpoint inhibitor” is defined in paragraphs [0044] and [0045].

In some embodiments, the immune checkpoint inhibitor administered in combination with the ATR inhibitor is selected from a PD-1 inhibitor, a PD-L1 inhibitor and a CTLA-4 inhibitor.

In some embodiments the immune checkpoint inhibitor may be a PD-1 inhibitor.

In some embodiments the immune checkpoint inhibitor may be a PD-L1 inhibitor.

In some embodiments the immune checkpoint inhibitor may be a CTLA-4 inhibitor.

In some embodiments the immune checkpoint inhibitor may be selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, BMS-986189, ipilimumab and tremelimumab.

In some embodiments the immune checkpoint inhibitor may be the PD-L1 inhibitor durvalumab or an antigen-binding fragment thereof. In some embodiments the immune checkpoint inhibitor may be durvalumab.

Durvalumab is a human monoclonal antibody directed against human PD-L1 that is capable of blocking the binding of PD-L1 to both the PD1 and CD80 receptors. Disclosure related to durvalumab can be found in U.S. Pat. Nos. 8,779,108 and 9,493,565, which are incorporated herein by reference.

Durvalumab and antigen-binding fragments thereof for use in the treatments provided herein comprises a heavy chain and a light chain or a heavy chain variable region and a light chain variable region. In some embodiments, durvalumab or antigen-binding fragment thereof for use in the treatments provided herein comprises a light chain variable region comprising the amino acid sequence of SEQ ID NO: 1 and a heavy chain variable region comprising the amino acid sequence of SEQ ID NO: 2. In some embodiments, durvalumab or antigen-binding fragment thereof for use in the treatments provided herein comprises a heavy chain variable region and a light chain variable region, wherein the heavy chain variable region comprises the Kabat-defined CDR1, CDR2, and CDR3 sequences of SEQ ID NOs: 3-5, and wherein the light chain variable region comprises the Kabat-defined CDR1, CDR2, and CDR3 sequences of SEQ ID NOs: 6-8. Those of ordinary skill in the art would easily be able to identify Chothia-defined, Abm-defined or other CDR definitions known to those of ordinary skill in the art. In some embodiments, durvalumab or antigen-binding fragment thereof for use in the treatments provided herein comprises the variable heavy chain and variable light chain CDR sequences of the 2.14H9OPT antibody as disclosed in U.S. Pat. Nos. 8,779,108 and 9,493,565, which are herein incorporated by reference in their entirety.

In some embodiments the immune checkpoint inhibitor may be durvalumab which is administered on every 4 weeks from cycle 1 day 1.

In some embodiments the immune checkpoint inhibitor may be durvalumab which is administered in an amount of 1500 mg.

In some embodiments the immune checkpoint inhibitor may be durvalumab which is administered in an amount of 1500 mg once every 4 weeks (28 days) from cycle 1 day 1 (Q28D).

In some embodiments the CTLA-4 inhibitor is tremelimumab or antigen-binding fragment thereof. Tremelimumab or antigen-binding fragment thereof for use in the treatments provided herein comprises a heavy chain and a light chain or a heavy chain variable region and a light chain variable region. In some embodiments, tremelimumab or antigen-binding fragment thereof for use in the treatments provided herein comprises a light chain variable region comprising the amino acid sequence of SEQ ID NO: 9 and a heavy chain variable region comprising the amino acid sequence of SEQ ID NO:10. In some embodiments, tremelimumab or antigen-binding fragment thereof for use in the treatments provided herein comprises a heavy chain variable region and a light chain variable region, wherein the heavy chain variable region comprises the Kabat-defined CDR1, CDR2, and CDR3 sequences of SEQ ID NOs:11-13, and wherein the light chain variable region comprises the Kabat-defined CDR1, CDR2, and CDR3 sequences of SEQ ID NOs:14-16. Those of ordinary skill in the art would easily be able to identify Chothia-defined, Abm-defined or other CDR definitions known to those of ordinary skill in the art. In some embodiments, tremelimumab or antigen-binding fragment thereof for use in the treatments provided herein comprises the variable heavy chain and variable light chain CDR sequences of the 11.2.1 antibody as disclosed in U.S. Pat. No. 6,682,736, which is herein incorporated by reference in its entirety.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “antigen-binding fragment” refers to a portion of an intact antibody and/or refers to the antigenic determining variable domains of an intact antibody. It is known that the antigen-binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

### Melanoma

“Cancer” is used synonymously with tumour and lesion in this specification. Cancer may include primary cancer as well as secondary cancers and metastases. The tumours may be detectable or non-detectable, e.g. micro metastases.

The “treatment of melanoma”, “treating melanoma” and similar terms encompass treating an existing melanoma cancer and/or preventing melanoma cancer. In some embodiments, treatment may be conducted after one or more symptoms have developed. In other embodiments, treatment may be conducted in the absence of symptoms. For example, treatment of a susceptible individual may begin prior to the onset of symptoms (e.g. due to a history of disease and/or considering genetic or other susceptibility factors). Treatment may also be continued after symptoms have resolved, for example to present or delay their recurrence

In some embodiments the treatment of melanoma or treating melanoma may mean treating and preventing melanoma.

In some embodiments the treatment of melanoma or treating melanoma may mean treating melanoma.

In some embodiments the treatment of melanoma or treating melanoma may mean preventing melanoma.

Mucosal and acral melanoma are known to be particularly difficult histologic subtypes of melanoma to treat. However, responses in these subtypes were observed in patients treated with the treatments described herein.

In some embodiments melanoma may be cutaneous melanoma.

In some embodiments melanoma may be cutaneous anal melanoma.

In some embodiments melanoma may be acral melanoma.

In some embodiments melanoma may be mucosal melanoma.

In some embodiments melanoma may be early stage, actively progressing, advanced (for example locally advanced), invasive, metastatic and/or drug-resistant melanoma.

In some embodiments melanoma may be locally advanced melanoma.

In some embodiments melanoma may be advanced and/or metastatic melanoma.

In some embodiments melanoma may be advanced melanoma.

In some embodiments melanoma may be locally advanced and/or metastatic melanoma.

In some embodiments melanoma may be metastatic melanoma.

In some embodiments melanoma may be invasive melanoma.

In some embodiments melanoma may be Stage IV melanoma.

In some embodiments melanoma may be unresectable melanoma. In some embodiments melanoma may be Stage III unresectable melanoma.

### Patient Selection

In some embodiments melanoma cancer may be ATM deficient.

When a melanoma cancer is “ATM deficient”, the cancer cells express less ATM protein than a normal, non-cancerous cell of the same type. For example, the cancer cells may express ≤5%, ≤10%, ≤20%, ≤30%, ≤40%, ≤50%, ≤60%, ≤70%, ≤80%, ≤90% or <100% of the total ATM protein expressed by a normal cell of the same type when analysed by IHC protein staining of the total ATM protein typically expressed by a normal cell of the same type when analysed by IHC protein staining. ATM deficient melanoma cancer cells may also comprise a biallelic deleterious mutation in their ATM gene.

In some embodiments melanoma cancer may be ARID1A deficient.

When a melanoma cancer is “ARID1A deficient”, the cancer cells express less ARID1A protein than a normal, non-cancerous cell of the same type. For example, the cancer cells may express ≤5%, ≤10%, ≤20%, ≤30%, ≤40%, ≤50%, ≤60%, ≤70%, ≤80%, ≤90% or <100% of the total ARID1A protein typically expressed by a normal cell of the same type when analysed by IHC protein staining. ARID1A deficient melanoma cancer cells may also comprise a mutation in the ARID1A gene (for example a loss of function mutation such as a nonsense mutation).

In some embodiments melanoma may be ATM deficient melanoma.

In some embodiments melanoma may be ARID1A deficient melanoma.

Since the current standard of care for melanoma is first-line immunotherapy, the combination described herein may be administered to patients as a second or third line of therapy.

In some embodiments the combination is administered to a patient as second line therapy i.e. after the patient has failed on prior immunotherapy.

In some embodiments, the combination is administered to a patient as third line therapy.

Patients receiving the combination as third line therapy may have received treatment with BRAF and MEK inhibitors, such as dabrafenib and trametinib, prior to treatment with immunotherapy.

### Clinical Properties

In one embodiment there is provided an ATR inhibitor such as AZD6738 in combination with an immune checkpoint inhibitor such as durvalumab for use in the treatment of melanoma, where the ATR inhibitor and the immune checkpoint inhibitor are administered to a patient who has previously received immunotherapy and treatment with the combination achieves an objective response rate between 10% and 50%, between 10% and 40%, between 10% and 35%, between 20% and 35%, between 25% and 40%, between 30% and 35%, greater than 10%, greater than 20%, greater than 30%, greater than 40%, greater than 50%, greater than 60% or greater than 70%.

“Objective response rate” is the percentage of patients with measurable disease at baseline and achieved at least 1 response, i.e. a complete or partial response to treatment according to RECIST.

The “RECIST criteria” (for example the RECIST 1.1 criteria) are set out at the site https://recist.eortc.org/ and described in Eur. J. Cancer 2016, 6, Pages 132-137.

In one embodiment there is provided an ATR inhibitor such as AZD6738 in combination with an immune checkpoint inhibitor such as durvalumab for use in the treatment of melanoma, where the ATR inhibitor and the immune checkpoint inhibitor are administered to a patient who has previously received immunotherapy and treatment with the combination achieves a clinical benefit rate between 25% and 100%, between 25% and 90%, between 40% and 80% or between 50% and 70%.

“Clinical benefit rate” is the objective response rate added to the % of patients whose best response was stable disease at the first scan, i.e. who had absence of disease progression at first scan (complete response+partial response+stable disease).

In one embodiment there is provided an ATR inhibitor such as AZD6738 in combination with an immune checkpoint inhibitor such as durvalumab for use in the treatment of melanoma, where the ATR inhibitor and the immune checkpoint inhibitor are administered to a patient who has previously received immunotherapy and treatment with the combination achieves a progression free survival of greater than 3 months, greater than 4 months, greater than 5 months, greater than 6 months, preferably greater than 7 months.

“Progression free survival” or “PFS” is the length of time during and after the treatment that a patient lives without the disease worsening. PFS may be determined using the Kaplan-Meier method.

In one embodiment there is provided an ATR inhibitor such as AZD6738 in combination with an immune checkpoint inhibitor such as durvalumab for use in the treatment of melanoma, where the ATR inhibitor and the immune checkpoint inhibitor are administered to a patient who has previously received immunotherapy and treatment with the combination achieves an overall survival of greater than 10 months, greater than 11 months, greater than 12 months, greater than 13 months, preferably greater than 14 months.

“Overall survival” or “OS” is the length of time from the start of treatment that a patient is still alive.

In one embodiment there is provided an ATR inhibitor such as AZD6738 in combination with an immune checkpoint inhibitor such as durvalumab for use in the treatment of melanoma, where the ATR inhibitor and the immune checkpoint inhibitor are administered to a patient who has previously received immunotherapy and treatment with the combination achieves a duration of response of at least 3 months, at least 4 months, at least 5 months, preferably at least 6 months.

“Duration of response” or “DoR” is the length of time that a tumour continues to respond to treatment without the cancer growing or spreading.

In one embodiment there is provided a combination of an ATR inhibitor such as AZD6738 and an immune checkpoint inhibitor such as durvalumab for use in the treatment of melanoma, where the combination is administered to a patient who has previously received immunotherapy, and treatment with the combination does not cause any serious side-effects in a melanoma patient.

In some embodiments serious side-effects may be defined as grade 4 or 5 adverse events.

“Grade 4 or 5 adverse events” can be classified according to the common terminology criteria for adverse events (CTCAE).

### Kits

In one aspect there is provided a kit comprising:
a) a first pharmaceutical composition comprising an ATR inhibitor and a pharmaceutically acceptable excipient;
b) a second pharmaceutical composition comprising an immune checkpoint inhibitor and a pharmaceutically acceptable excipient; and
c) instructions for the use of the use of the first and second pharmaceutical compositions in the treatment of melanoma in a patient who has previously received immunotherapy.

In one embodiment the ATR inhibitor may AZD6738.

In one embodiment the immune checkpoint inhibitor may be selected from a PD-1 inhibitor, a PD-L1 inhibitor and a CTLA-4 inhibitor.

In some embodiments the immune checkpoint inhibitor may be selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, BMS-986189, ipilimumab and tremelimumab.

In some embodiments the immune checkpoint inhibitor may be durvalumab.

In one embodiment the ATR inhibitor is AZD6738 and the immune checkpoint inhibitor is durvalumab.

### Specific Embodiments

In one embodiment there is provided an ATR inhibitor for use in the treatment of melanoma, where the ATR inhibitor is AZD6738 which is administered in combination with an immune checkpoint inhibitor to a patient who has previously received therapy with an immune checkpoint inhibitor.

In one embodiment there is provided an ATR inhibitor for use in the treatment of melanoma, where the ATR inhibitor is AZD6738 which is administered in combination with a PD-L1 inhibitor to a patient who has previously received therapy with an immune checkpoint inhibitor selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, BMS-986189, ipilimumab and tremelimumab.

In one embodiment there is provided an ATR inhibitor for use in the treatment of melanoma, where the ATR inhibitor is AZD6738 which is administered in combination with durvalumab to a patient who has previously received therapy with an immune checkpoint inhibitor selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, BMS-986189, ipilimumab and tremelimumab.

In one embodiment there is provided an ATR inhibitor for use in the treatment of melanoma, where the ATR inhibitor is AZD6738 which is administered in combination with durvalumab to a patient who has previously received therapy with an immune checkpoint inhibitor selected from pembrolizumab and nivolumab.

### Examples

### Abbreviations

AE—Adverse event
ATM—Ataxia Telangiectasia Mutated
ATR—Ataxia-Telangiectasia and Rad3-related protein
BD—twice daily
CRF—Case Report Form (electronic/paper)
CTCAE—Common Terminology Criteria for Adverse Events
ctDNA—Circulating Tumor DNA
DNA—Deoxyribonucleic acid
ECG—Electrocardiogram
MSI—Microsatallite Instable Tumors
MSS—Microsatallite Stable Tumors
SAE—Serious adverse event

### Clinical Trial Protocol

Introduction: This study was a multi-centre phase II study of AZD6738 in combination with durvalumab in patients with refractory solid tumor. The tumor types studied were: Gastric cancer 2^{nd} line of treatment and beyond (outside the scope of the present specification); and metastatic melanoma who had progressed after standard therapy. Patients received AZD6738 plus durvalumab combination regimen. The study was fully recruited to a total of 60 patients (30 in each tumor type) but follow up is ongoing. AZD6738 was administered at 240 mg BD on days 15 to 28 in a 28-day cycle. Durvalumab was administered at 1500 mg fixed dose every 4 weeks from cycle 1 day 1. Tumour evaluation using modified RECIST 1.1 was conducted at screening (within 28 days prior to first dose) and every 8 weeks relative to the date of first dose, up to week 40, then every 12 weeks until objective disease progression (within a window of +/−7 days of the scheduled date). Study treatment was continued until objective disease progression (unless other criteria for treatment discontinuation are met). Patients may have continued AZD6738 plus durvalumab beyond progression (according to modified RECIST 1.1), at the discretion of the investigator if they were clinically benefiting from the treatment and they did not meet any other discontinuation criteria. However, they were not permitted to continue either AZD6738 or durvalumab as monotherapy. If a patient discontinued study treatment prior to disease progression, they should have continued to be assessed using modified RECIST 1.1 until disease progression and then followed up for survival. Assessments for survival were made every 8 weeks following objective disease progression. The details of first and subsequent therapies for cancer, after discontinuation of treatment, was collected. The imaging modalities used for modified RECIST 1.1 assessment was CT or MRI scans of chest, abdomen and pelvis. Modified RECIST 1.1 scans were analysed by the investigator on site. Patients were also requested to provide tumour samples from the primary or metastatic tumours on progression. Sample provision was not optional, subject to a specific consent, and will aid understanding of resistance mechanisms. However, if biopsy site was not feasible, the protocol allowed waiving the rebiopsy procedure.

### Primary Objectives and Outcome Measures:

| Primary Objective | Outcome Measure |
|---|---|
| To evaluate the safety and | AEs/SAEs |
| tolerability of Drug AZD6738 | Vital signs |
| in combination with durvalumab | Collection of clinical |
| | chemistry/haematology parameters |
| To investigate the efficacy of | Objective response rate (ORR) by |
| AZD6738 when given in combination | modified RECIST 1.1 |
| with durvalumab in patients with | |
| refractory solid tumor patients | |

### Secondary Objectives and Outcome Measures:

| Secondary Objective | Outcome Measure |
|---|---|
| To assess secondary | Clinical benefit rate (CR + PR + SD) |
| measures of clinical | Duration of response |
| efficacy | Overall survival (OS) and progression- |
| | free survival |
| | (PFS) calculated by Kaplan-Meier |
| | method |
| Planned subgroup analyses | ATM deficient and ATM proficient in |
| | terms of RR, PFS, OS |
| | MSI-high and MSS in terms of RR, PFS, |
| | OS |
| | PDL1 high and low in terms of RR, PFS, |
| | OS |
| | Other exploratory analysis; Tumor |
| | sample, ctDNA (Resistant mechanism) |
| To investigate the efficacy | Objective response rate (ORR) by |
| of AZD6738 in subgroup | modified |
| | RECIST 1.1 in Overall population |
| | including planned subgroup. |

### Tertiary Objectives and Outcome Measures:

| | |
|---|---|
| Teriary Objective | Outcome Measure |

Tumor

| | |
|---|---|
| To investigate predictive markers for | To collect and store diagnostic and/or |
| sensitivity or resistance to AZD6738 in | archival tumour samples AND any fresh |
| combination with durvalumab that may be | tumour biopsies (pre-treatment and at |
| observed in tumour (archival and/or fresh | progression) for potential future |
| frozen) from patients treated with AZD6738 in | exploratory research into factors that |
| combination with durvalumab to determine if | may influence development and/or |
| there are any other biomarkers that may | response to AZD6738 in combination |
| predict sensitivity or resistance to treatment. | with durvalumab. Next generation |
| A progression tumor specimen will also be | sequencing from these samples and/or |
| collected for exploratory analysis to determine | other protein based exploratory |
| mechanisms of acquired resistance to | analysis will be carried out. Baseline |
| treatment. | fresh biopsies are mandatory. |

cfDNA

| | |
|---|---|
| To investigate predictive markers and acquired | Genomic approaches including but not |
| resistance to AZD6738 and/or durvalumab | limited to next generations sequencing |
| that may be observed in circulating free DNA | and PCR will be used for future |
| (cfDNA) extracted from plasma preparation of | exploratory research into factors that |
| blood samples from patients treated with | can predict sensitivity to AZD6738 in |
| AZD6738 in combination with durvalumab. | combination with durvalumab. Samples |
| | collected during the course of |
| | treatment may be used to elucidate |
| | mechanisms of resistance to |
| | treatments. |

cytokines

| | |
|---|---|
| To investigate cytokines or other circulating | To collect and store plasma and/or |
| soluble factors in peripheral blood as | PBMC samples for analysis by analytical |
| pharmacodynamic endpoints for AZD6738 | methods e.g. SIMOA, Singulex, |
| and/or durvalumab | Luminex, MSD. Pharmacodynamic |
| | effects of investigational products may |
| | be assessed vs. pharmacokinetics to |
| | establish dose/PK/PD relationships. |

Target subject population: There were two cohorts for this study although Cohort A is outside of the scope of the present specification:

Cohort A: Patients with refractory gastric cancer who had failed secondary chemotherapy treatments for advanced disease were enrolled. Patients must have had imaging confirmed progression on previous chemotherapy for gastric cancer treatment with at least one measurable lesion per modified RECIST 1.1. Previous adjuvant/neoadjuvant chemotherapy was allowed, if completed more than 6 months prior to starting the 1st line treatment. Prior exposure to AZD6738 was not allowed.

Cohort B: Patients with metastatic melanoma patients who had failed prior anti-PD(L)1 were enrolled. Anti-PD(L)1 therapy should have been the immediate prior regimen before study entry.

Duration of treatment: Patients continued to receive study treatment, until they demonstrated objective disease progression (determined by modified RECIST 1.1) or until they met any other discontinuation criteria. There was no maximum duration of treatment with AZD6738 and durvalumab. Patients could continue with AZD6738 and durvalumab beyond objective disease progression (determined by modified RECIST 1.1) at the discretion of the investigator if they were clinically benefiting from the treatment and they did not meet any other discontinuation criteria.

Patients continued to receive treatment with AZD6738 and durvalumab provided that the treatment was tolerable and there was evidence of clinical benefit (as judged by the investigator) and secure supply of medication. Upon confirmation of objective disease progression, or treatment discontinuation criteria are met, both durvalumab and AZD6738 were discontinued. If either durvalumab and/or AZD6738 were deemed intolerable (as judged by the investigator) so that discontinuation of either agent was deemed in the patient's best interest despite dose interruptions, dose modification and initiation of supportive treatments, both durvalumab and AZD6738 were discontinued and the patient withdrawn from the study. Patients were not permitted to continue either durvalumab or AZD6738 as monotherapy. There was no maximum duration of treatment with AZD6738 and durvalumab.

Duration of treatment Investigational product, dosage and mode of administration AZD6738 was administered at 240 mg BD on days 15 to 28 in a 28-day cycle. Durvalumab was administered at 1500 mg every 4 weeks from cycle 1 day 1.

### Statistical Methods

Cohort A (gastric cancer): The primary endpoint of the study was ORR (independently analyzed for cohort A and cohort B). The sample size was calculated by use of a two-stage minimax Simon's design to control the type I error at 5% for null hypothesis that, for arm, the true response was 15% or below and to have 90% of power if the true response was 40% or higher. 16 evaluable patients were to be treated in the first stage. If 2 or fewer response were observed in the first stage, the arm would have been stopped. If at least 3 responses were observed in the first stage, 11 additional evaluable patients were to be entered onto the second stage. At the final analysis, the null hypothesis was to be rejected if at least 8 responses were observed in 27 evaluable patients. RR was reported with its exact 95% Cl.

Cohort B (melanoma): The sample size was calculated by use of a two-stage minimax Simon's design to control the type I error at 5% for null hypothesis that, for arm, the true response was 15% or below and to have 90% of power if the true response was 40% or higher. 16 evaluable patients were to be treated in the first stage. If 2 or fewer responses were observed in the first stage, the arm was to be stopped. If at least 3 responses were observed in the first stage, 11 additional evaluable patients were to be entered onto the second stage. At the final analysis, the null hypothesis was to be rejected if at least 8 responses were observed in 27 evaluable patients. RR was reported with its exact 95% Cl.

Inclusion Criteria: Patients were eligible to be included in the study only if all the following inclusion criteria and none of the exclusion criteria applied:
1. Provision of fully informed consent prior to any study specific procedures.
2. Patients ≥18 years of age
3. Patient with ATM deficient or ATM proficient through IHC. ATM expression status was assessed prospectively. Minimum numbers of each patient group (ATM proficient and deficient) were required for analysis, therefore central prospective screening was to be deployed to ensure this was achieved.
4. Body weight >30 kg
5. Cohort A: Confirmed histological or cytological diagnosis of gastric adenocarcinoma (including GEJ) that was at an advanced stage and that had progressed following who have failed secondary chemotherapy treatments (confirmed by imaging)
6. Cohort B: Confirmed melanoma (metastatic) who had progressed to prior anti-PD(L)1 therapy (immediate prior regimen)
7. Had the presence of measurable disease as defined by the Response Evaluation Criteria in Solid Tumors (modified RECIST) version 1.1 which was suitable for accurate repeated measurements.
8. Provision of tumor sample (from either a resection or biopsy) for ATM IHC and other exploratory biomarker
9. Patients were willing and able to comply with the protocol for the duration of the study including undergoing treatment and scheduled visits and examinations.
10. ECOG performance status 0-1 with no deterioration between screening and the first dose of study treatment
11. Patients had a life expectancy 3 months from proposed first dose date.
12. Patients had a washout period of 3 weeks for any prior therapy prior to the start of study drug. The following intervals between the end of the prior treatment and first dose of study drug must have been observed: 4 weeks for radiotherapy (patients who receive palliative radiation for nontarget lesions need not have had a 4 week washout period and could be enrolled immediately); patients may have received a stable dose of bisphosphonates or denusomab as long as these were started at least 4 weeks prior to treatment; ≥4 weeks for major surgery; 7 days for minor surgical procedures; ≥14 days (or 5 half lives whoever is longest) for any investigational product.
13. Patients had acceptable bone marrow, liver and renal function measured within 28 days prior to administration of study treatment as defined below:
   Haemoglobin ≥9.0 g/dL (transfusion not permitted within 14 days of study medication)
   Absolute neutrophil count (ANC) ≥1.5×109/L
   Platelet count ≥100×109/L (transfusion not permitted within 14 days of study medication
   Total bilirubin 1.5×institutional upper limit of normal (ULN)
   AST (SGOT)/ALT (SGPT) ≤2.5×institutional upper limit of normal unless liver metastases are present in which case it must be ≤5×ULN
   Serum creatinine ≤1.5×institutional ULN
   Glomerular filtration rate <45 mL/min as assessed by standard methodology at the investigating centre
   Haematuria: +++ on microscopy or dipstick
14. Female patients of childbearing potential had a negative pregnancy test (urine or serum), were not breastfeeding and were using adequate contraceptive measures. Female patients used a highly effective contraceptive measure from screening until 90 days after the last dose of drug. All methods of contraception (except for total abstinence) were used in combination with the use of a condom by a male sexual partner for intercourse. Female patients had evidence of non-childbearing potential by fulfilling one of the following criteria at screening:
   a. Post-menopausal women defined as aged more than 50 years and amenorrhoeic for at least 12 months following cessation of all exogenous hormonal treatment.
   b. Documentation of irreversible surgical sterilisation by hysterectomy, bilateral oophorectomy, or bilateral salpingectomy, but not tubal ligation.
   c. Amenorrhoeic for 12 months and serum follicle-stimulating hormone (FSH), lutenizing hormone (LH) and plasma oestradiol levels in the postmenopausal range for the institution
15. For the duration of the study and for 1 week after the last study drug administration, sexually active male patients must have been willing to use barrier contraception i.e. condoms with all sexual partners. Where the sexual partner was a ‘women of child-bearing potential’ who was not using effective contraception, men used a condom (with spermicide) during the study and for 6 months after the last dose of a study drug.
16. Mandatory biopsy during the screening window prior to dosing and at progression (fresh frozen will be mandatory if clinically feasible)

Exclusion Criteria: Patients did not enter the study if any of the following exclusion criteria were fulfilled:
1. Diagnosis of ataxia telangiectasia.
2. Any previous treatment with ATR inhibitors, DNA—damage repair inhibitors
3. Any gastrointestinal condition that would preclude adequate absorption of AZD6738 including but not limited to inability to swallow oral medication, refractory nausea and vomiting, chronic gastrointestinal diseases or previous significant bowel resection, intestinal obstruction or CTCAE grade 3 or grade 4 upper GI bleeding within 4 weeks before the enrollment.
4. Active or prior documented autoimmune or inflammatory disorders (including IBD [e.g. Chohn's disease, ulcerative colitis or diverticulitis], SLE, sarcoidosis syndrome, tuberculosis, Wegener syndrome, myasthenia gravis, Graves' disease, rheumatoid arthritis, hypophysitis, uveitis, history of primary immunodeficiency or HIV infection, known hepatitis B or hepatitis C infection, history of organ transplant that requires use of immunosuppressives, glomerulonephritis, nephritic syndrome, Fanconi Syndrome or renal tubular acidosis within the past 2 years prior to the start of treatment. The following are exceptions to this criterion:
   Subjects with vitiligo or alopecia, hypothyroidism (eg, following Hashimoto syndrome) stable on hormone replacement or psoriasis not requiring systemic treatment; patients with coeliac disease controlled by diet alone and patients without active disease in the last 5 years may be included after consultation with Chief Investigator.
5. Untreated central nervous system (CNS) metastatic disease, leptomeningeal disease, or cord compression. Note: Any subjects previously treated for CNS metastases that were asymptomatic, radiographically and neurologically stable for at least 4 weeks and did not require corticosteroids (of any dose) for symptomatic management for at least 4 weeks prior to the first dose of treatment were not excluded.
6. Patients with second primary cancer, except: adequately treated non-melanoma skin cancer, curatively treated in-situ cancer of the cervix, or other solid tumours curatively treated with no evidence of disease for 3 years.
7. Current or prior use of immunosuppressive medication within 4 weeks prior to the first dose of durvalumab, with the exceptions of intranasal, topical, and inhaled corticosteroids; systemic corticosteroids at physiologic doses not to exceed a dose >10 mg prednisone/day or equivalent)
8. Patient in receipt of any live attenuated vaccination within 30 days prior to study entry or within 30 days of receiving study therapy.
9. Receiving or having received, concomitant medications, herbal supplements, and/or foods that significantly modulate P450 3A4 (CYP3A4) or Pgp activity (washout periods of 5 half-lives). Note these include common azole antifungals, macrolide antibiotics and other medications.
10. Patient with any of the following cardiac criteria:
   Mean QT interval corrected for heart rate (QTc) ≥470 ms calculated from 3 electrograms (ECGs) using Friderecia's correction
   Any clinically important abnormalities in fhythm, conduction or morphology of resting ECG e.g. complete left bundle branch block, third degree heart block, second degree heart block.
   Any factors that increase the risk of QTc prolongation or risk of arrhythmic events such as heart failure, hypokalaemia, congenital long QT syndrome, family history of long QT syndrome or unexplained sudden death under 40 years of age or concomitant medication known to prolong the QT interval
   Uncontrolled hypotension: systolic BP<90 mmHg and/or diastolic BP 60 mmHg or clinically relevant orthostatic hypotension, including a fall in blood pressure of >20 mmHg
   Atrial fibrillation with a ventricular rate >100 bpm on ECG at rest
   Symptomatic heart failure (NYHA grade II-IV)
   Known reduced LVEF <55%
   Prior or current cardiomyopathy
   Severe valvular heart disease
   Uncontrolled angina (Canadian Cardiovascular Society grade II-IV despite medical therapy)
   Stroke or transient ischaemic attack in the last 6 months prior to screening
   Acute coronary syndrome within 6 months prior to starting treatment
11. Ophthalmological conditions as follows: Intra-ocular pressure >21 mmHg, or uncontrolled glaucoma (irrespective of intra-ocular pressure), Current or past history of central serous retinopathy or retinal vein occlusion
12. Any evidence of severe or uncontrolled systemic disease, including active infection (requiring antibiotics, antifungals or antivirals), diabetes type I and II, uncontrolled seizures, bleeding diatheses, severe COPD, severe Parkinson's disease.
13. Any unresolved toxicity NCI CTCAE Grade ≥2 from previous anticancer therapy with the exception of alopecia, vitiligo, Major surgical procedure (as defined by the Investigator) within 28 days prior to the first dose of IP. Note: Local surgery of isolated lesions for palliative intent was acceptable.
14. Female patients who were pregnant or breastfeeding or male or female patients of reproductive potential who were not willing to employ effective birth control from screening to 90 days after the last dose of durvalumab monotherapy.
15. Known allergy or hypersensitivity to any of the study drugs or any of the study drug excipients.
16. Restrictions
   For durvalumab:
      Patients should not have donated blood while participating in this study or for at least 90 days following the last infusion of durvalumab.
      Immunosuppressive medications including, but not limited to systemic corticosteroids at doses beyond 10 mg/day of prednisone or equivalent, methotrexate, azathioprine, and tumour necrosis factor alpha blockers were prohibited. Use of immunosuppressive medications for the management of study drug-related AEs and in patients with contrast allergies was acceptable. In addition, use of inhaled and intranasal corticosteroids was permitted.
      Live attenuated vaccines within 30 days of durvalumab dosing (ie, 30 days prior to the first dose, during treatment with durvalumab and for 30 days post discontinuation of durvalumab). Inactivated viruses, such as those in the influenza vaccine, are permitted.
      The following restrictions applied whilst the patient was receiving study treatment and for the specified times before and after:
         Female patient of child-bearing potential
   Females of childbearing potential who were sexually active with a non-sterilized male partner were to use at least 1 highly effective method of contraception from the time of screening and must have agreed to continue using such precautions for 180 days after the last dose of durvalumab+any drug combination therapy. Non-sterilized male partners of a female patient were to use male condom plus spermicide throughout this period. Cessation of birth control after this point should have been discussed with a responsible physician. Not engaging in sexual activity for the total duration of the drug treatment and the drug washout period was an acceptable practice; however, periodic abstinence, the rhythm method, and the withdrawal method were not acceptable methods of birth control. Female patients also refrained from breastfeeding throughout this period.
      Male patients with a female partner of childbearing potential Non-sterilized males who were sexually active with a female partner of childbearing potential were to use a male condom plus spermicide from screening through 180 days after receipt of the final dose of durvalumab+any drug combination therapy. Not engaging in sexual activity was an acceptable practice; however, occasional abstinence, the rhythm method, and the withdrawal method were not acceptable methods of contraception. Male patients refrained from sperm donation throughout this period.
   Female partners (of childbearing potential) of male patients were also to use a highly effective method of contraception throughout this period.
      Females of childbearing potential were defined as those who were not surgically sterile (i.e., bilateral tubal ligation, bilateral oophorectomy, or complete hysterectomy) or post-menopausal.
      Women were considered post-menopausal if they had been amenorrheic for 12 months without an alternative medical cause. The following age-specific requirements apply:
   Women <50 years of age were considered post-menopausal if they had been amenorrheic for 12 months or more following cessation of exogenous hormonal treatments and if they had luteinizing hormone and follicle-stimulating hormone levels in the post-menopausal range for the institution or underwent surgical sterilization (bilateral oophorectomy or hysterectomy).
   Women ≥50 years of age were considered post-menopausal if they had been amenorrheic for 12 months or more following cessation of all exogenous hormonal treatments, had radiation-induced menopause with last menses >1 year ago, had chemotherapy-induced menopause with last menses >1 year ago, or underwent surgical sterilization (bilateral oophorectomy, bilateral salpingectomy or hysterectomy).
   For AZD6738:
   The following restrictions applied while the patient was receiving study treatment and for the specified times before and after: Patients must fast (water to drink only) from at least 2 hours prior to taking a dose to at least 1 hour post-dose for all doses.
      If vomiting occurred shortly after AZD6738 was swallowed, the dose should only have been replaced if all of the intact tablets could be counted and then treatment resumed with the following scheduled dose. The scheduled dose could be taken up to 2 hours after the scheduled dose time. If greater than 2 hours, the missed dose should not have been taken and patient continued with next dose at allotted time.
      If no clinically significant blood pressure (BP) changes were seen following the single dose, then no restrictions with regard to driving were required
      For all patients, it was not recommended to consume grapefruit juice or Seville oranges (including marmalade, juice, etc) while participating in the study.
   Male patients:
      Men were to use a condom (with spermicide) during the study, and for 1 week after the last dose of study drug, with all sexual partners
      Where a sexual partner of a male participant was a ‘woman of child-bearing potential’ who was not using effective contraception, men were to use a condom (with spermicide) during the study and for 6 months after the last dose of a study drug
      Must not donate sperm for 6 months after the last dose of study drug
   Female patients:
      Contraceptives that were prone to drug-drug interactions may not have been effective due to a potential CYP3A4 interaction with AZD6738. Contraception used must therefore have included a condom and one of:
         Medroxyprogesterone injections (eg, Depo-provera)
         Intrauterine Device (IUD)
         Levonorgestrol Intrauterine System (eg, Mirena)
         Tubal occlusion
         Vasectomised partner

Efficacy assessments: This study assessed the efficacy of AZD6738 when given in combination with durvalumab in patients with advanced gastric cancer as a second line regimen.

Modified RECIST 1.1 criteria was used to assess patient response to treatment by determining PFS and ORR. The modified RECIST 1.1 guidelines for measurable, non-measurable, target and non-target lesions and the objective tumour response criteria (complete response, partial response, stable disease or progression of disease). The methods of assessment of tumour burden used at baseline, CT or MRI scans of chest, abdomen and pelvis must be used at each subsequent follow-up assessment. Following the baseline assessment, efficacy for all patients was assessed by objective tumour assessments every 8 weeks relative to date of first dose, until week 40, at which time assessments were carried out every 12 weeks until objective disease progression as defined by modified RECIST 1.1.

If a patient discontinues treatment (and/or receives a subsequent cancer therapy) prior to progression then the patient was to still continue to be followed until objective disease progression as defined by modified RECIST 1.1.

Categorisation of objective tumour response assessment was based on the modified RECIST 1.1 criteria of response: CR (complete response), PR (partial response), SD (stable disease) and PD (progression of disease). Target lesion (TL) progression was calculated in comparison to when the tumour burden was at a minimum (i.e. smallest sum of diameters previously recorded on study). In the absence of progression, tumour response (CR, PR, SD) was calculated in comparison to the baseline tumour measurements obtained before starting treatment.

For patients with non-measurable disease only at baseline, categorisation of objective tumour response assessment was based on the modified RECIST 1.1 criteria of response: CR, PD and Non CR/Non PD. If the investigator was in doubt as to whether progression has occurred, particularly with response to NTL (non-target lesion) or the appearance of a new lesion, it was advisable to continue treatment until the next scheduled assessment or sooner if clinically indicated and reassess the patient's status. If repeat scans confirm progression, then the date of the initial scan should be declared as the date of progression.

Any other sites at which new disease is suspected should also be appropriately imaged. If an unscheduled assessment was performed and the patient had not progressed, every attempt was to be made to perform the subsequent assessments at their scheduled visits. To achieve ‘unequivocal progression’ on the basis of non-target disease, there must have been an overall level of substantial worsening in non-target disease such that, even in presence of SD or PR in target disease, the overall tumour burden had increased sufficiently to merit discontinuation of therapy. A modest ‘increase’ in the size of one or more non-target lesions is usually not sufficient to quality for unequivocal progression status.

### Safety Assessments

Blood and urine samples for determination of clinical chemistry, hematology, and urinalysis were taken at the times indicated in the assessment schedules and as clinically indicated. Clinical laboratory safety tests, including serum pregnancy tests, were to be performed in a licensed clinical laboratory according to local standard procedures. Sample tubes and sample sizes may vary depending on the laboratory method used and routine practice at the site. Pregnancy tests may be performed at the site using a licensed test (urine or serum pregnancy test). Abnormal clinically significant laboratory results should have been repeated as soon as possible (preferably within 24 to 48 hours). Additional safety samples were collected if clinically indicated at the discretion of the Investigator. The date, time of collection, and results (values, units, and reference ranges) were recorded on the appropriate CRF. Other safety tests were performed at screening including assessment for hepatitis B surface antigen, hepatitis C antibodies, and HIV antibodies.

Laboratory safety assessments included:

Full haematology assessments:
(a) Haemoglobin.
(b) Red blood cells (RBC).
(c) Platelets.
(d) Mean cell volume (MCV).
(e) Mean cell haemoglobin concentration (MCHC).
(f) Mean cell haemoglobin (MCH).
(g) WBC.
(h) Absolute differential white cell count (neutrophils, lymphocytes, monocytes, eosinophils and basophils) and absolute neutrophil count or segmented neutrophil count and Band forms were to be performed at each visit and when clinically indicated. If absolute differentials not available % differentials were to be provided.
(i) Coagulation:
   Activated partial thromboplastin time (APTT) was to be performed at baseline and if clinically indicated
   International normalised ratio (INR) was to be performed at baseline and if clinically indicated unless the patient is receiving warfarin. Patients taking warfarin were able to participate in this study; however, it was recommended that prothrombin time (INR and APTT) be monitored carefully at least once per week for the first month, then monthly if the INR was stable.
(j) Biochemistry assessments for safety:
   Sodium
   Potassium
   Calcium
   Magnesium
   Creatinine
   Total bilirubin
   Gamma glutamyl transferase (GGT)
   ALP
   AST
   ALT
   Urea or blood urea nitrogen (BUN)
   Total protein
   Albumin
   Lactic dehydrogenase (LDH).

Urinalysis: Urinalysis was performed at screening and if clinically indicated. Microscopic analysis was performed by the hospital's local laboratory if required.

Physical examination: as per paragraph [00176]

ECG: ECGs were performed at screening, baseline, once on combination treatment and as clinically indicated throughout the study and were as described in paragraph [00178].

Vital signs: Height was assessed at screening only. Weight was assessed at screening and as clinically indicated at any other time. Any changes in vital signs were recorded as an AE, if applicable. Supine BP and pulse rate were measured using a semi-automatic BP recording device with an appropriate cuff size, after patient had rested for at least 10 minutes and was assessed on Day 1 of each cycle (within a window of +/−7 days of the scheduled date). If being assessed within 7 days before first dose and meets the stated eligibility criteria (if applicable), it was not needed to be repeated on Day 1 of Cycle 1. The date and time of collection and measurement was recorded on the appropriate eCRF. Body temperature was measured in degrees Celsius using an automated thermometer at screening and as clinically indicated at any other time. The date of collection and measurement was recorded on the appropriate eCRF.

Other Safety Assessments: Two pregnancy tests on blood or urine samples were performed for pre-menopausal women of childbearing potential one within 28 days prior to the start of study treatment. Tests were performed by the hospital's local laboratory. If results were positive the patient was ineligible/was discontinued from the study. In the event of a suspected pregnancy during the study, the test was to be repeated.

Concomitant Medications: any medications (other than those excluded by the clinical trial protocol) that were considered necessary for the subject's welfare and would not interfere with the trial medication were to be given at the investigator's discretion. The investigator was to record all concomitant medication taken by subject during the trial, from the date of signature of informed consent, in the appropriate section of the CRF.

The following were not permitted during the trial:
(a) Prophylactic granulocyte colony stimulating factor or granulocyte macrophage colony stimulating factor.
(b) Concomitant use of known potent CYP3A4 inhibitors such as ketoconazole, itraconazole, ritonavir, indinavir, saquinavir, telithromycin, clarithromycin and nelfinavir.
(c) To avoid potential reductions in exposure due to drug interactions, the following CYP3A4 inducers were to be avoided: Phenytoin, rifampicin, rifapentine, rifabutin, carbamazepine, phenobarbitone, nevirapine, modafinil and St John's Wort.
(d) Traditional Chinese Medicine with an approved anticancer indication.

### Clinical Trial Results: AZD6738 and Durvalumab

30 melanoma patients (10 cutaneous, 10 acral and 10 mucosal) were enrolled in a clinical trial according to the above protocol and all had resistance to PD-L1 inhibitor. 30 patients were evaluable for efficacy, with the following results observed: 9 confirmed partial responses (30%), 11 stable disease (37%) and 10 disease progression (33%). The overall confirmed response rate was 30% (95% Cl 15%-49%). The disease control rate (DCR) was 67% (20 of 30 patients; Cl 47%-83%). Duration of response (DoR) of greater than 6 months was 67% (95% Cl 28%-88%) with a median duration of 10.0 months (95% Cl 3.2—NA). Median progression free survival (PFS) was 7.0 months (95% Cl 1.8-11.8; 67% mature) whilst the median overall survival (OS) was 14.0 months (95% Cl 9.9—NA; 47% mature).

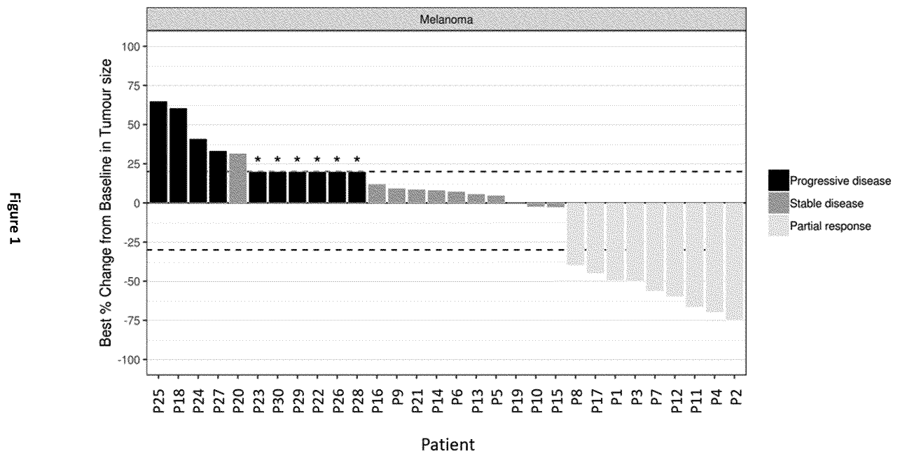

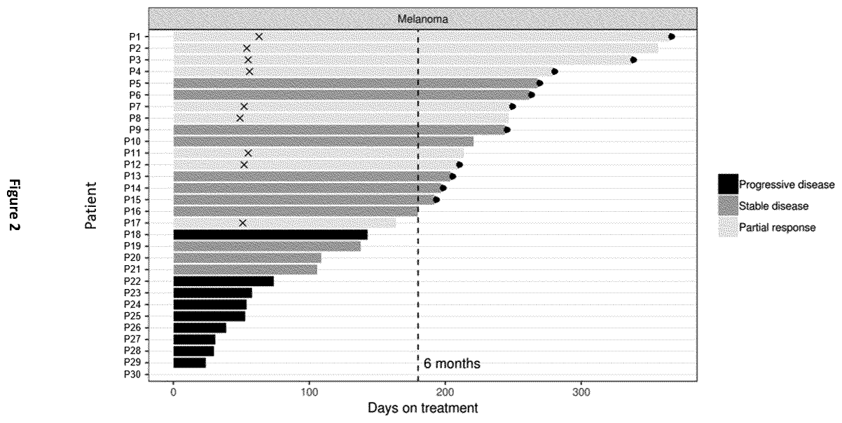

FIGS. 1 and 2 present the clinical trial data of the AZD6738 and durvalumab combination in graphical form.

Tables 1 and 2 set out the clinical characteristics and the resistance status of the patients in the clinical trials for the ceralasertib (AZD6738) and durvalumab combination. Responses were seen irrespective of whether patients had primary or secondary resistance. Furthermore, although it is known to be harder to treat patients with primary resistance, a greater proportion of responses were seen in the patients receiving ceralaserib and durvalumab who had primary resistance compared to patients with secondary resistance. Responses were seen in all the melanoma subtypes treated (acral, mucosal and cutaneous). Responses were also seen across patients with high/low baseline LDH (lactate dehydrogenase), low PD-L1 expression and regardless of BRAF mutant or wildtype.

TABLE 1
Clinical Characteristics of the Patients

| | Ceralasertib + durvalumab |
|---|---|
| | (total n = 30; responders n = 9) |
| Responses in IO refractory | 4 had PD as best response on prior IO |
| patients | 4 had SD as best response on prior IO |
| | 1 had PR as best response on prior IO |
| Type of resistance to prior | Primary n = 9 of which 4 responses |
| PD-(L)1 | (44%) |
| | Secondary n = 18 of which 2 responses |
| | (11%) |
| | Early relapse in the adjuvant setting |
| | n = 2 of which 2 responses (100%) |
| | Non-evaluable (<6 weeks drug |
| | exposure): 1 who responded (100%) |
| Responses in all 3 | 3 Cutaneous |
| melanoma subtypes | 2 Acral |
| | 4 Mucosal |
| Responses in patients with | 6/16 with ≤ULN |
| high baseline LDH, a poor | 2/11 with >ULN and ≤ULN |
| prognostic indicator | 1/3 with >2xULN |
| Responses in patients with | 6 had PD-L1 expression = 0-1% |
| low PD-L1 expression | 1 had PD-L1 expression = 10% |
| (investigator assessed) | 1 had PD-L1 expression = 50% |
| | 1 patient unknown |
| Brain mets | 1 PR had brain mets, 8 without brain |
| | mets |
| Liver mets | 4/9 (44%) |
| No clear association with | 3 responders (2 mucosal) had BRAF |
| BRAF, NRAS or KRAS status | mutation (V600E) |

IO: immunotherapy;
SD: stable disease;
PD: progressive disease;
PR: partial response;
LDH: lactate dehydrogenase;
ULN: upper limit of normal

TABLE 2
Primary and Secondary Resistance

| | | | | PD after | |
|---|---|---|---|---|---|
| | | | Early | discon- | NE (not |
| | Primary | Secondary | relapse | tinuation | evaluable) |
| Ceralasertib + | 9 (30%) | 18 (60%) | 2 (7%) | N/A | 1 (3%) |
| durvalumab | | | | | |
| ORR | 4 (44%) | 2 (11%) | | | |
| DCR | 4 (44%) | 12 (67%) | | | |
| PFS | 3.6 mo | 7.3 mo | | | |
| OS | 9.9 mo | 14.6 mo | | | |

ORR: objective response rate;
DCR: disease control rate;
PFS: progression free survival;
OS: overall survival

TABLE 3
Disclosed Sequences

| SEQ ID NO: | Sequence | Description |
|---|---|---|
| 1 | EIVLTQSPGTLSLSPGERATLSCRASQRVSSSYLAWYQQK | Light chain variable |
| | PGQAPRLLIYDASSRATGIPDRFSGSGSGTDFTLTISRLEP | domain of durvalumab |
| | EDFAVYYCQQYGSLPWTFGQGTKVEIK | |
| 2 | EVQLVESGGGLVQPGGSLRLSCAASGFTFSRYWMSWV | Heavy chain variable |
| | RQAPGKGLEWVANIKQDGSEKYYVDSVKGRFTISRDNA | domain of durvalumab |
| | KNSLYLQMNSLRAEDTAVYYCAREGGWFGELAFDYWG | |
| | QGTLVTVSS | |
| 3 | GFTFSRYWMS | CDRH1 of durvalumab |
| 4 | NIKQDGSEKYYVDSVKG | CDRH2 of durvalumab |
| 5 | EGGWFGELAFDY | CDRH3 of durvalumab |
| 6 | RASQRVSSSYLA | CDRL1 of durvalumab |
| 7 | DASSRAT | CDRL2 of durvalumab |
| 8 | QQYGSLPWT | CDRL3 of durvalumab |
| 9 | PSSLSASVGDRVTITCRASQSINSYLDWYQQKPGKAPKL | Light chain variable |
| | LIYAASSLQSGVPSRFSGSGSGTDFTLTISSLQPEDFATYY | domain of |
| | CQQYYSTPFTFGPGTKVEIKRTVAAPSVFIFPPSDEQLKS | tremelimumab |
| | GTASVVCLLNNFYPREAKV | |
| 10 | GVVQPGRSLRLSCAASGFTFSSYGMHWVRQAPGKGLE | Heavy chain variable |
| | WVAVIWYDGSNKYYADSVKGRFTISRDNSKNTLYLQM | domain of |
| | NSLRAEDTAVYYCARDPRGATLYYYYYGMDVWGQGTT | tremelimumab |
| | VTVSSASTKGPSVFPLAPCSRSTSESTAALGCLVKDYFPE | |
| | PVTVSWNSGALTSGVH | |
| 11 | GFTFSSYGMH | CDRH1 of |
| | | tremelimumab |
| 12 | VIWYDGSNKYYADSV | CDRH2 of |
| | | tremelimumab |
| 13 | TAVYYCARDPRGATLYYYYYGMDV | CDRH3 of |
| | | tremelimumab |
| 14 | RASQSINSYLD | CDRL1 of tremelimumab |
| 15 | AASSLQS | CDRL2 of tremelimumab |
| 16 | QQYYSTPFT | CDRL3 of tremelimumab |

1. (canceled)

2. A method of treating melanoma in a human or animal patient, who has previously received immunotherapy, in need of such treatment, comprising administering to said patient an effective amount of an ATR inhibitor in combination with an effective amount of an immune checkpoint inhibitor.

3. (canceled)

4. The method according to claim 2, wherein the ATR inhibitor is selected from the group consisting of AZD6738, M6620 (berzosertib), BAY-1895344, EPT-46464, VE-821 and VX-970.

5. The method according to claim 4, wherein the ATR inhibitor is AZD6738.

6. The method according to claim 2, wherein the immune checkpoint inhibitor is a PD-1 inhibitor, a PD-L1 inhibitor or a CTLA-4 inhibitor.

7. The method according to claim 6, wherein the immune checkpoint inhibitor is a PD-L1 inhibitor.

8. The method according to claim 7, wherein the PD-L1 inhibitor is durvalumab or an antigen-binding fragment thereof.

9. The method according to claim 2, wherein the melanoma is selected from the group consisting of cutaneous, acral and mucosal melanoma.

10. The method according to claim 2, wherein the melanoma is advanced melanoma.

11. The method according to claim 2, wherein the melanoma is metastatic melanoma.

12. The method according to claim 5, wherein AZD6738 is administered on days 15 to 28 of a 28-day cycle.

13. The method according to claim 5, wherein AZD6738 is administered on days 1 to 7 of a 28-day cycle.

14. The method according to claim 5, wherein total daily dose of AZD6738 is between 30 mg and 500 mg.

15. The method according to claim 14, wherein the total daily dose of AZD6738 is administered twice daily in 240 mg doses.

16. The method according to claim 14, wherein the total daily dose of AZD6738 is administered twice daily in 160 mg doses.

17. The method according to claim 8, wherein durvalumab is administered in an amount of 1500 mg once every 4 weeks from cycle 1 day 1.

18. The method according to claim 2, wherein the previous immunotherapy comprises treatment with an immune checkpoint inhibitor.

19. The method according to claim 18, wherein the previous immunotherapy comprises treatment with an immune checkpoint inhibitor is selected from a PD-1 inhibitor, PD-L1 inhibitor and a CTLA-4 inhibitor.

20. The method according to claim 19, wherein the previous immunotherapy comprises treatment with an immune checkpoint inhibitor is selected from the group consisting of pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, BMS-986189, ipilimumab and tremelimumab.

21. The method according to claim 20, wherein the previous immunotherapy comprises treatment with pembrolizumab or nivolumab.

22. The method according to claim 2, wherein the patient has primary resistance to immunotherapy.

23. The method according to claim 2, wherein the patient has acquired resistance to immunotherapy.

24. The method according to claim 2, wherein the patient received immunotherapy as adjuvant therapy.

25. The method according to claim 2, wherein the treatment achieves an objective response rate greater than 30%.

26. The method according to claim 2, wherein progression free survival is greater than 3 months.

27. The method according to claim 2, where overall survival is greater than 10 months.

28. The method according to claim 2, wherein duration of response is at least 3 months.

29. A kit comprising:
a. a first pharmaceutical composition comprising an ATR inhibitor and a pharmaceutically acceptable excipient;
b. a second pharmaceutical composition comprising an immune checkpoint inhibitor and a pharmaceutically acceptable excipient; and instructions for the use of the use of the first and second pharmaceutical compositions in the treatment of melanoma in a patient who has previously received immunotherapy.


SEQUENCE LISTING (16 sequences)

SEQ ID NO: 1 - protein, 108 aa, Artificial Sequence
  Light chain variable domain of durvalumab
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Artificial Sequence
  Heavy chain variable domain of durvalumab
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 10 aa, Artificial Sequence
  CDRH1 of durvalumab
GFTFSRYWMS

SEQ ID NO: 4 - protein, 17 aa, Artificial Sequence
  CDRH2 of durvalumab
NIKQDGSEKYYVDSVKG

SEQ ID NO: 5 - protein, 12 aa, Artificial Sequence
  CDRH3 of durvalumab
EGGWFGELAFDY

SEQ ID NO: 6 - protein, 12 aa, Artificial Sequence
  CDRL1 of durvalumab
RASQRVSSSYLA

SEQ ID NO: 7 - protein, 7 aa, Artificial Sequence
  CDRL2 of durvalumab
DASSRAT

SEQ ID NO: 8 - protein, 9 aa, Artificial Sequence
  CDRL3 of durvalumab
QQYGSLPWT

SEQ ID NO: 9 - protein, 139 aa, Artificial Sequence
  Light chain variable domain of tremelimumab
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 10 - protein, 167 aa, Artificial Sequence
  Heavy chain variable domain of tremelimumab
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 11 - protein, 10 aa, Artificial Sequence
  CDRH1 of tremelimumab
GFTFSSYGMH

SEQ ID NO: 12 - protein, 15 aa, Artificial Sequence
  CDRH2 of tremelimumab
VIWYDGSNKYYADSV

SEQ ID NO: 13 - protein, 24 aa, Artificial Sequence
  CDRH3 of tremelimumab
TAVYYCARDPRGATLYYYYYGMDV

SEQ ID NO: 14 - protein, 11 aa, Artificial Sequence
  CDRL1 of tremelimumab
RASQSINSYLD

SEQ ID NO: 15 - protein, 7 aa, Artificial Sequence
  CDRL2 of tremelimumab
AASSLQS

SEQ ID NO: 16 - protein, 9 aa, Artificial Sequence
  CDRL3 of tremelimumab
QQYYSTPFT